(19) United States
(12) Patent Application Publication
(10) Pub. No.: US 2009/0297615 A1
(43) Pub. Date: Dec. 3, 2009

(54) NANOPARTICLES, METHODS OF MAKING SAME AND CELL LABELING USING SAME

(75) Inventors: Yi-Xiang Wang, Hong Kong (CN); Cham-Fai Leung, Hong Kong (CN); Ling Qin, Hong Kong (CN)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Chinese University of Hong Kong, Hong Kong (CN)

(21) Appl. No.: 12/400,591

(22) Filed: Mar. 9, 2009

Related U.S. Application Data
(60) Provisional application No. 61/056,170, filed on May 27, 2008.

(51) Int. Cl. A61K 47/48; B32B 9/00; B05D 7/00; C12Q 1/02; A61K 9/16

(52) U.S. Cl. 424/490; 428/402; 428/403; 428/404; 428/407; 427/215; 427/221; 427/220; 435/29; 514/769; 977/773

(57) ABSTRACT

There are disclosed polyhedral superparamagnetic nanoparticles and methods for making and using the nanoparticles. There are also disclosed coated and functionalized forms of the nanoparticles, methods of using nanoparticles and methods of treatment using nanoparticles.

This application claims the benefit of U.S. Provisional Application No. 61/056,170 filed May 27, 2008, the entire contents of which are hereby incorporated herein by reference.

### FIELD

The subject matter disclosed generally relates to nanoparticles, methods of making nanoparticles and cell labelling.

### BACKGROUND

Superparamagnetic iron oxide (SPIO) is an MRI contrast agent commonly used for the purpose of stem cell labelling, in the form of nanoparticles. Such nanoparticles may be coated with dextran and may be used clinically for intracellular labelling. PCT/US03/00051, Gaw and Josephson, filed Jan. 2, 2003 describes amine functionalized superparamagnetic nanoparticles and the coating of nanoparticles with dextran. UK patent GB2415374 to Persoons et al, published Dec. 28, 2005 describes the use of nano nanoparticles for the delivery of bioactive substances. “Silica- and Alkoxysilane-Coated Ultrasmall Superparamagnetic Iron Oxide nanoparticles: A Promising Tool To Label Cells for Magnetic Resonance Imaging” Chunfu Zhang et al Nov. 30, 2006 Journal of the American Chemical Society, describes the use of silica coated nanoparticles for cell labelling.

### SUMMARY

The SPIO nanoparticles presented in this disclosure may be suitable for labelling of cells. In embodiments the nanoparticles may comprise an iron oxide core, may comprise a coating and may be functionalized.

A polyhedral superparamagnetic nanoparticle comprising a metal oxide.

In embodiments the polyhedral superparamagnetic nanoparticle may have a shape selected from the group consisting of: primitive cubic, body-centered cubic, face-centered cubic, primitive tetragonal, body-centered tetragonal, primitive orthorhombic, body-centered orthorhombic, single face-centered orthorhombic, multiple face-centered orthorhombic, primitive monoclinic, single face-centered monoclinic, primitive triclinic, single face-centered hexagonal, and rhombohedral, monohedral, parallelohedral, dihedral, dishpenoid, prism, pyramid, dipyramid, trapezohedron, scalenohedron, rhombohedron, and tetrahedron.

In embodiments there is disclosed a superparamagnetic nanoparticle according to any of the other embodiments wherein the metal is selected from the group consisting of: Cobalt, Titanium, Manganese, Magnesium, Nickel, Copper, Zinc, Vanadium, Gold, Palladium, Platinum and Iron.

In embodiments there is disclosed a superparamagnetic nanoparticle according to any of the other embodiments wherein the nanoparticle is coated and the coating comprises a material selected from the group consisting of silica, dextran, polyethylene glycol, polylactic acid, polyglycolic acid, poly(N-isopropylacrylamide) (PNIPAM), hydroxyapatite, layered double hydroxide and alginate.

In embodiments there is disclosed a superparamagnetic nanoparticle according to any of the other embodiments wherein the nanoparticle is functionalized with functional groups selected from the group consisting of amine, ammonium, alkylamine, dialkylamine, amide, hydroxyl, ether, carboxyl, ester, thiol, thioether, alkene, and alkyne.

In embodiments there is disclosed a superparamagnetic nanoparticle according to any of the other embodiments wherein the nanoparticle is made by a process comprising heating the superparamagnetic metal oxide to above a temperature of greater than 50 C, 80 C, 100 C, 120 C, 140 C, 160 C, 180 C, 200 C, or greater than 200 C.

In embodiments there is disclosed a nanoparticle according to any of the other embodiments wherein the nanoparticle comprises superparamagnetic iron oxide.

In embodiments there is disclosed a nanoparticle according to any of the other embodiments wherein the nanoparticle is substantially cubic.

In embodiments there is disclosed a nanoparticle of wherein the nanoparticle has a diameter in a range selected from the group consisting of: between about 1 nm and about 500 nm, between about 1 nm and about 300 nm, between about 1 nm and about 150 nm, between about 1 nm and about 50 nm, between about 1 nm and about 10 nm, between about 3 and about 10 nm and between about 5 nm and about 8 nm.

In embodiments there is disclosed a nanoparticle of wherein the nanoparticle has a diameter between about 3 nm and about 10 nm.

In embodiments there is disclosed a nanoparticle according to any of the other embodiments wherein the metal oxide is comprised in a core and wherein the nanoparticle further comprises a silica coating associated with the core.

In embodiments there is disclosed a nanoparticle according to any of the other embodiments wherein nanoparticle has a plurality of reactive primary amino groups.

In embodiments there is disclosed a nanoparticle according to any of the other embodiments wherein the metal oxide is comprised in a core and wherein the nanoparticle further comprises a silica coating associated with the core.

In embodiments there is disclosed a nanoparticle according to any of the other embodiments wherein nanoparticle has a plurality of reactive primary amino groups.

In embodiments there is disclosed a nanoparticle according to any of the other embodiments wherein the nanoparticle is conjugated with a group that comprises a molecule selected from the group consisting of: nucleic acid, protein, antibody, lectin, carbohydrate, antibiotic, pharmaceutical, anti-cancer drug, and wound healing drug.

In embodiments there is disclosed a method for synthesizing a polyhderal superparamagnetic metal oxide nanoparticle, the method comprising the steps of: making amorphous nanoparticles of the superparamagnetic metal oxide and heating the amorphous nanoparticles to more than about 100 C for more than about 8 hours.

In embodiments there is disclosed a method for synthesizing a polyhderal superparamagnetic metal oxide nanoparticle, the method comprising the steps of: making amorphous nanoparticles of the superparamagnetic metal oxide and autoclaving the amorphous nanoparticles.

In embodiments there is disclosed the method according to any of the other embodiments wherein the nanoparticle has a shape selected from the group consisting of: primitive cubic, body-centered cubic, face-centered cubic, primitive tetragonal, body-centered tetragonal, primitive orthorhombic, body-centered orthorhombic, single face-centered orthorhombic, multiple face-centered orthorhombic, primitive monoclinic, single face-centered monoclinic, primitive triclinic, single face-centered hexagonal, and rhombohedral, monohedral, parallelohedral, dihedral, dishpenoid, prism, pyramid, dipyramid, trapezohedron, scalenohedron, rhombohedron, and tetrahedron.

In embodiments there is disclosed the method according to any of the other embodiments wherein the metal is selected from the group consisting of: Cobalt, Titanium, Manganese, Magnesium, Nickel, Copper, Zinc, Vanadium, Gold, Palladium, Platinum and Iron.

In embodiments there is disclosed the method according to any of the other embodiments further comprising coating the nanoparticles with a material comprising an ingredient selected from the group consisting of silica, dextran, polyethylene glycol, polylactic acid, polyglycolic acid, poly(N-isopropylacrylamide) (PNIPAM), hydroxyapatite, layered double hydroxide and alginate.

In embodiments there is disclosed the method according to any of the other embodiments further comprising functionalizing the nanoparticle with functional groups selected from the group consisting of amine, ammonium, alkylamine, dialkylamine, amide, hydroxyl, ether, carboxyl, ester, thiol, thioether, alkene, and alkyne.

In embodiments there is disclosed the method according to any of the other embodiments further comprising coprecipitating a mixture of a first metal ion and a second metal ion to make the amorphous nanoparticles.

In embodiments there is disclosed the method according to any of the other embodiments comprising heating the precipitate to at least about 100 C for at least about 10 hours under pressure.

In embodiments there is disclosed the method according to any of the other embodiments where the first metal ion and the second metal ion are different valency states of the same metal.

In embodiments there is disclosed the method according to any of the other embodiments wherein the firs metal ion is an Fe(II) ion and the second metal ion is an Fe(III) ion.

In embodiments there is disclosed the method according to any of the other embodiments further comprising coating the nanoparticle with silica.

In embodiments there is disclosed the method according to any of the other embodiments further comprising coating the nanoparticle with amine groups.

pharmaceutical composition comprising the nanoparticle according to in association with a pharmaceutically acceptable carrier.

A method of labelling a cell comprising:
(a) labelling the cell with a polyhedral superparamagnetic nanoparticle; and
(b) detecting the nanoparticle.

In embodiments there is disclosed the method according to any of the other embodiments wherein the nanoparticle has a shape selected from the group consisting of: primitive cubic, body-centered cubic, face-centered cubic, primitive tetragonal, body-centered tetragonal, primitive orthorhombic, body-centered orthorhombic, single face-centered orthorhombic, multiple face-centered orthorhombic, primitive monoclinic, single face-centered monoclinic, primitive triclinic, single face-centered hexagonal, and rhombohedral, monohedral, parallelohedral, dihedral, dishpenoid, prism, pyramid, dipyramid, trapezohedron, scalenohedron, rhombohedron, and tetrahedron.

In embodiments there is disclosed the method according to any of the other embodiments wherein the metal is selected from the group consisting of: Cobalt, Titanium, Manganese, Magnesium, Nickel, Copper, Zinc, Vanadium, Gold, Palladium, Platinum and Iron.

In embodiments there is disclosed the method according to any of the other embodiments wherein the nanoparticle is coated and the coating comprises a material selected from the group consisting of silica, dextran, polyethylene glycol, polylactic acid, polyglycolic acid, poly(N-isopropylacrylamide) (PNIPAM), hydroxyapatite, layered double hydroxide and alginate.

In embodiments there is disclosed the method according to any of the other embodiments wherein the nanoparticle is functionalized with functional groups selected from the group consisting of amine, ammonium, alkylamine, dialkylamine, amide, hydroxyl, ether, carboxyl, ester, thiol, thioether, alkene, and alkyne.

In embodiments there is disclosed the method according to any of the other embodiments wherein the nanoparticle comprises a superparamagnetic iron oxide core and a silica coating having primary amino groups.

In embodiments there is disclosed the method according to any of the other embodiments wherein the nanoparticle is between about 1 nm and 25 nm in diameter.

In embodiments there is disclosed the method according to any of the other embodiments wherein the cell is selected from the group consisting of a mesenchymal cell, a stem cell, a nerve cell, muscle cell, a malignant cell, a Stem cell, a Nerve cell, a Tumoral cell, a Osteoblast, a Osteocyte, a Osteoclast, a Chondroblast, a Chondrocyte, a Myocyte, a Adipocyte, a Fibroblast, a Tendon cell, a Podocyte, a Juxtaglomerular cell, a Intraglomerular mesangial cell/Extraglomerular mesangial cell, a Kidney proximal tubule brush border cell, a Macula densa cell, a Gastric chief cell, a Parietal cell, a Goblet cell, a Paneth cell, a Enteroendocrine cells, a Enterochromaffin cell, a APUD cell, a Hepatocyte, a Kupffer cell, a Myocardiocyte, a Pericyte, a Pneumocyte, a Type I pneumocyte, a Type II pneumocyte, a Clara cell, a Goblet cell, a glial cell, an Astrocyte, a Microglia, a Thyroid epithelial cell, a Parafollicular cell, a Parathyroid chief cell, a Chromaffin cell, a lymphoid: B/T T cell, a Natural killer cell, a granulocyte, a Basophil granulocyte, an Eosinophil granulocyte, a Neutrophil granulocyte, a Hypersegmented neutrophil, a Monocyte, a Macrophage, a Red blood cell, a Reticulocyte, a Mast cell, a Thrombocyte, a Megakaryocyte, and a Dendritic cell.

In embodiments there is disclosed the method according to any of the other embodiments wherein the cell is a cell in a mammalian body.

In embodiments there is disclosed the method according to any of the other embodiments wherein the method comprises a step selected from the group consisting of: labelling the cell in vivo, labelling the cell ex vivo, delivering the nanoparticles orally, topically, transdermally, intraperitoneally, intraocularly, intracranially, intracerebroventricularly, intracerebralyl, intravaginally, intrauterinely, nasally, rectally, parenterally, subcutaneously, intravascularly, observing the cells in vivo, observing the cells ex vivo, localizing the nanoparticles, localizing the nanoparticles using a magnetic field, and separating cells containing nanoparticles from cells not containing nanoparticles.

In embodiments there is disclosed a method of treating a subject in need of the treatment, the treatment comprising administering to the subject in association with a pharmaceutically acceptable experiment a superparamagnetic nanoparticle according to any of the other embodiments conjugated with a therapeutically effective group.

In embodiments there is disclosed the use of a superparamagnetic nanoparticle according to any of the embodiments to manufacture a medicament.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

FIG. 1A is a TEM view of nanoparticles according to an embodiment.

FIG. 1B is an EDX spectrum of the nanoparticles according to FIG. 1A.

FIG. 1C is an XRD spectrum of the nanoparticles according to FIG. 1A.

FIG. 1D is a VSM spectrum of the nanoparticles according to FIG. 1A.

FIG. 2A is an optical microscopy image of nanoparticle distribution in a population of mesenchymal stem cells.

FIG. 2B is a higher power view of labelled cells from a portion of FIG. 2.

FIG. 3A is a 4,200× TEM image of mesenchymal stem cells labelled with nanoparticles of an embodiment.

FIG. 3B is a 11,500× TEM image of mesenchymal stem cells labelled with nanoparticles of an embodiment.

FIG. 3C is a 16,500× TEM image of mesenchymal stem cells labelled with nanoparticles of an embodiment.

FIG. 3D is a 160,000× TEM image of mesenchymal stem cells labelled with nanoparticles of an embodiment.

FIG. 4A is 100× optical microscope image of unlabelled rabbit bone marrow mesenchymal stem cells with osteogenic differentiation.

FIG. 4B is 100× optical microscope image of rabbit bone marrow mesenchymal stem cells labelled with nanoparticles according to an embodiment with osteogenic differentiation.

FIG. 4C is 200× optical microscope image of unlabelled rabbit bone marrow mesenchymal stem cells with adipogenic differentiation.

FIG. 4D is 200× optical microscope image of rabbit bone marrow mesenchymal stem cells labelled with nanoparticles according to an embodiment with adipogenic differentiation.

FIG. 4E is 200× optical microscope image of unlabelled rabbit bone marrow mesenchymal stem cells with chondrogenic differentiation.

FIG. 4F is 200× optical microscope image of rabbit bone marrow mesenchymal stem cells labelled with nanoparticles according to an embodiment with chondrogenic differentiation.

FIG. 5A is a series of gradient echo magnetic resonance images of pelleted mesenchymal stem cells labelled with SPIO-SiO₂ nanoparticles.

FIG. 5B is a series of gradient echo magnetic resonance images of pelleted mesenchymal stem cells labelled with SPIO-SiO₂—NH₂ nanoparticles.

FIG. 6A is a rabbit brain T2W 2D spin echo image in sagittal plane immediately post MSCs implantation.

FIG. 6B is rabbit brain of FIG. 6A 8 weeks post implantation with T2W 3D veno-BOLD imaging in sagittal plane.

FIG. 7A is SPIO-SiO₂—NH₂ nanoparticles labelled MSCs induced signal void on 2D gradient echo T2W images 2 days post-implantation in the brain

FIG. 7B is the same view as seen in FIG. 7A, but taken 12 weeks post MSCs implantation.

FIG. 8A is a view of SPIO-SiO₂—NH₂ nanoparticles labelled MSCs induced signal void on 2D gradient echo T2W images 2 days post-implantation in the left erector spinae.

FIG. 8B is the same view seen in FIG. 8A but taken 12 weeks post MSCs implantation.

FIG. 9A is a light microscope view of MSCs 3 week after labelling with SPIO-SiO₂—NH₂.

FIG. 9B is an equivalent view to FIG. 9A but shows MSCs 3 weeks after labelling with PEG-6000 coated SPIO.

FIG. 10. is a microscope view of 12 weeks post SPIO-SiO₂—NH₂ nanoparticles labelled MSCs implantation in the left erector spinae of a rabbit.

FIG. 10A is a light microscope view of MSCs 3 weeks after labelling with SPIO-SiO₂—NH₂.

FIG. 10B is an equivalent view to FIG. 10A but shows MSCs 3 weeks after labelling with SPIO-SiO₂—NH₂.

FIG. 11 is a TEM view of nanoparticles (Fe₃O₄—NH₂, without silica coating) according to an embodiment.

FIG. 12 is a TEM view of nanoparticles (MnFe₂O₄—NH₂, without silica coating) according to an embodiment.

### DETAILED DESCRIPTION OF THE PREFERRED EMBODIMENTS

Terms

In this disclosure the following terms have the meaning set forth below:

In this disclosure, unless otherwise indicated, all numbers expressing quantities or ingredients, measurement of properties and so forth used in the specification and claims are to be understood as being modified in all instances by the term “about”. Accordingly, unless indicated to the contrary or necessary in light of the context, the numerical parameters set forth in the disclosure are approximations that can vary depending upon the desired properties sought to be obtained by those skilled in the art utilizing the teachings of the present disclosure and in light of the inaccuracies of measurement and quantification. Without limiting the application of the doctrine of equivalents to the scope of the claims, each numerical parameter should at least be construed in light of the number of reported significant digits and by applying ordinary rounding techniques. Not withstanding that the numerical ranges and parameters setting forth the broad scope of the disclosure are approximations, their numerical values set forth in the specific examples are understood broadly only to the extent that this is consistent with the validity of the disclosure and the distinction of the subject matter disclosed and claimed from the prior art.

In this disclosure, the word “comprising” is used in a non-limiting sense to mean that items following the word are included, but items not specifically mentioned are not excluded. A reference to an element by the indefinite article “a” does not exclude the possibility that more than one of the elements is present, unless the context clearly requires that there be one and only one of the elements.

In this disclosure the recitation of numerical ranges by endpoints includes all numbers subsumed within that range including all whole numbers, all integers and all fractional intermediates (e.g., 1 to 5 includes 1, 1.5, 2, 2.75, 3, 3.80, 4, and 5 etc.).

In this disclosure the singular forms a “an”, and “the” include plural referents unless the content clearly dictates otherwise. Thus, for example, reference to a composition containing “a compound” includes a mixture of two or more compounds.

In this disclosure term “or” is generally employed in its sense including “and/or” unless the content clearly dictates otherwise.

In this disclosure the term “coat”, “coating”, “coated” or the like refers to a layer of material that partly or completely surrounds or encloses the superparamagnetic core of a nanoparticle. In embodiments the coating may be or may comprise silica, dextran, polyethylene glycol, polylactic acid, polyglycolic acid, poly(N-isopropylacrylamide) (PNIPAM), hydroxyapatite, layered double hydroxides and alginate or alginate or alginate In embodiments the coating may be a silica coating and may comprise layer of silica that envelops or partly envelops the magnetic core of a nanoparticle. The silica may be of any form, crystalline or amorphous and in embodiments it may be combined with other chemicals. In embodiments a silica coating may be achieved treating a metal oxide core with tetramethylorthosilicate (TMOS) for silica coating instead of tetraethylorthosilicate (TEOS). For silica-coated SPIO-SiO₂ nanoparticles, either TMO or TEOS can be used. For amine and silica coated SPIO-SiO₂—NH₂ nanoparticles, aminopropyl triethoxylsilane, aminopropyl trimethoxylsilane, aminobutyl triethoxylsilane, aminobutyl trimethoxylsilane, aminopentyl triethoxylsilane, aminopentyl trimethoxylsilane, and any other aminoalkyl trialkoxysilanes, which will be readily selected from by those skilled in the art.

In this disclosure the statement that a material or structure is “polyhedral” or “crystalline” or is in the form of a crystal or like statements, includes materials or structures that may have primitive, body centered and face centered forms and regular and irregular forms and includes primitive cubic, body-centered cubic, face-centered cubic, primitive tetragonal, body-centered tetragonal, primitive orthorhombic, body-centered orthorhombic, single face-centered orthorhombic, multiple face-centered orthorhombic, primitive monoclinic, single face-centered monoclinic, primitive triclinic, single face-centered hexagonal, and rhombohedral crystals and lattices and may be monohedral, parallelohedral, dihedral, dishpenoid, prism, pyramid, dipyramid, trapezohedron, scalenohedron, rhombohedron, or tetrahedron forms. In this disclosure the statement that a material adopts or may adopt any particular form of crystal or polyhedral structure includes forms which substantially adopt such structures but may include minor deviations from and irregularities in the adoption of such model structures.

In this disclosure the term “crystal” means a solid body having a characteristic internal structure and enclosed by substantially symmetrically arranged planar surfaces intersecting at definite and characteristic angles.

In this disclosure the term “polyhedron” means a solid figure having a plurality of faces.

In this disclosure the term “precipitate” means the preparation of a substance in solid from a solution by means of a reagent, or the solid or material thereby prepared and includes such prepared substance in solution prior to any settlement or separation from solution.

In this disclosure, unless the context clearly requires otherwise, “salts” means soluble salts. It will be understood that suitable metal salts for the precipitation of superparamagnetic nanoparticles and materials includes any conventional metal salts. A non limiting list of common examples of suitable salts may include sulphates, chlorides, phosphates, and nitrates which may be useable in different embodiments.

In this disclosure where valent forms or ions of a metal are indicated it is to be understood that in embodiments these may be comprised in the form of a salt. Thus by way of example Fe(II) represents divalent Fe ions which may be combined with any suitable salt anion or combination of salt anions.

In this disclosure, a nanoparticle means a material with a “core” of magnetic material which may in certain embodiments be enclosed in an outer coating layer of a different material. In embodiments the nanoparticle may have associated reactive primary amino groups or other groups either on the core or on the coating. These groups may be available for subsequent reactions, e.g., for the attachment of biomolecules. The nanoparticles may have an overall size less than about 100 nm, before conjugation to biomolecules. The overall diameter of the nanoparticles or the cores of the nanoparticles may be about 1 nm to 100 nm, about 1 to 50 nm, about 1 to 20 nm, about 5 to 15 nm, about 50 to 100 nm, or may be greater than about 1, 5,10, 20, 50, or 100 nm or less than about 100, 150, 100, 50, 40, 30, 20, 10, or 5 nm or the overall diameter may be up to or above about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nm. While a wide range of sizes is possible, in particular embodiments the nanoparticle may have a diameter in a range selected from the group comprising: between about 1 nm and about 500 nm, between about 1 nm and about 300 nm, between about 1 nm and about 150 nm, between about 1 nm and about 50 nm, between about 1 nm and about 10 nm, between about 3 and about 10 nm and between about 5 nm and about 8 nm. In embodiments, the average nanoparticle size (diameter) may be between about 5 and about 15 nanometers. In alternative embodiments the nanoparticles may be between about 1 and 2, 2 and 4, 5 and 7, 8 and 10, 10 and 12, 12 and 15, 16 and 18, 18 and 21 nm in diameter, or may be less than about 1 nanometer or greater than about 20 nanometer in diameter. Size can be determined by laser light scattering by atomic force microscopy or other suitable techniques.

Where the term “particle” is used herein it will be understood that unless the context clearly dictates otherwise, then this refers to and is interchangeable with a “nanoparticle” as herein defined.

In embodiments the nanoparticle or the nanoparticle core can be monodisperse (a single crystal of a magnetic material, e.g., metal oxide, such as superparamagnetic iron oxide, per nanoparticle) or polydisperse (a plurality of crystals, e.g., 2, 3, or 4, or more per nanoparticle). The metal oxides may be or may comprise crystals of about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nm or more in overall diameter.

In embodiments, the nanoparticles may be coated with a wide range of materials including but not limited to reactive, inert, amphiphilic, polar and non polar, biologically or chemically active materials, and in embodiments possible coatings may consist of or comprise silica, dextran, polyethylene glycol, polylactic acid, polyglycolic acid, alginate, poly(N-isopropylacrylamide) (PNIPAM), hydroxyapatite, layered double hydroxide and alginate. In further embodiments the nanoparticles may further comprise or may be associated or conjugated with a contrast agent suitable to enhance particular forms of detection, for example for use in MRI. In embodiments, the nanoparticles may comprise a superparamagnetic form of iron oxide. Superparamagnetic iron oxide is one of the highly magnetic forms (magnetite, non-stoichio-metric magnetite, gamma-ferric oxide) that may have a magnetic moment of greater than about 30 EMU/gm Fe at 0.5 Tesla and about 300 K. When magnetic moment is measured over a range of field strengths, it shows magnetic saturation at high fields and lacks magnetic remanence when the field is removed. In embodiments the magnetic metal oxide of a nanoparticle or a nanoparticle core may comprise Iron, Cobalt, Titanium, Manganese, Magnesium, Nickel, Copper, Zinc, Vanadium, Gold, Palladium, or Platinum, or alternative metals or mixtures of or comprising one or more of them. Those skilled in the art will readily select between such metals and combinations of metals for particular purposes taking into account their physical properties, cost, toxicity and the like. In embodiments the nanoparticles may be delivered to an organism or tissue or cells using a medical device, and may be injected or may be applied orally, topically, transdermally, intraperitoneally, intraocularly, intracranially, intracerebroventricularly, intracerebralyl, intravaginally, intrauterinely, orally, nasally, rectally or parenterally (e.g., intravenous, intraspinal, subcutaneous or intramuscular) subcutaneously, intravascularly, by catheter, or by any other conventional method. In embodiments the nanoparticles or cells labeled therewith may be delivered to an organism or tissue or cells using a medical device, and may be injected parenterally, subcutaneously, intravascularly, by catheter or by any other conventional methods. In alternative embodiments the nanoparticles or cells labelled therewith may be delivered systemically, or locally, or their delivery may be restricted to a particular range of cell types or characteristics, all using a range of conventional methods that will be readily understood and implemented by those skilled in the art. In embodiments the delivery of the nanoparticles may comprise or may be directed or localized by internally or externally applied magnetic fields. In embodiments the nanoparticles may be made or rendered water soluble using various techniques, such as surface modification. Those skilled in the art will readily understand and implement a range of modifications to nanoparticles such as the additional encapsulation of individual nanoparticles with molecules, or polymer, or biodegradable polymers such as polylacetic acid (PLA), polyglycolic acid (PGA), PLGA (PLA-co-PGA), poly(N-isopropylacrylamide) (PNIPAM), dextran, hydroxyapatite, layered double hydroxide and alginate and such as functionalization of the nanoparticle with desirable functional groups.

In this disclosure the statement that a nanoparticle is “functionalized” means that the nanoparticle (with or without a coating) has been treated to bear functional groups, in embodiments such functional groups may be or may include amine, ammonium, alkylamine, dialkylamine, amide, hydroxyl, ether, carboxyl, ester, thiol, thioether, alkene, alkyne NH₂, N⁺H3, NHR, NR₂, C(O)NHR, OH, OR, COOH, COOR, SH, SR, C═CH₂, C═CHR, C═CR₂, C≡CH, C≡CR, aromatics (where R=includes straight and branched alkyl chains, ring structures and combinations of the foregoing).

In this disclosure the term “conjugate” or “conjugation” or the like of nanoparticles means linking of the nanoparticles to chemicals or materials and the terms “bioconjugate” or “bioconjugation” and like terms indicates conjugating the nanoparticles to chemicals, molecules, complexes, structures, biomolecules, bioactive chemicals and the like. In embodiments the nanoparticles may be conjugated with a range of pharmaceuticals, chemicals or materials, for particular purposes and in particular embodiments conjugated pharmaceuticals may comprise anti-cancer drugs. Suitable biomolecules may include but are not limited to proteins, nucleic acids, DNA, RNA, carbohydrates, lipids, antibodies, lectins, streptavidin, proteins, enzymes, hormones, vitamins, ligands, receptors, pharmaceuticals, Doxorubicin, Taxol, Traditional Chinese Medicines, and all manner of biological or biologically active molecules. Those skilled in the art will readily select suitable conjugates, including bioconjugates and suitable methods of conjugation to suit particular purposes. Generally conjugation may be accomplished by means of covalent linkages but in embodiments it may be carried out using other forms of linkage. In embodiments the conjugates or the coating of the nanoparticles may be useable to target the nanoparticles to specific cell types and locations or to modify their properties for specific purposes. Those skilled in the art will readily understand how to make suitable modifications to achieve these purposes.

In this disclosure the term “magnetic” means materials of high positive magnetic susceptibility.

In this disclosure the statement that a formulation may be administered orally refers to its use in any suitable dosage form which may include capsules, cachets, pills, tablets, lozenges (optionally using a flavored basis, such as sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes, and the like, each containing a predetermined amount of a therapeutic agent as an active ingredient. A compound may also be administered as a bolus, electuary or paste. Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof. Powders are prepared by comminuting the compound to a suitable fine size and mixing with a similarly comminuted pharmaceutical carrier such as an edible carbohydrate, as, for example, starch or mannitol.

In this disclosure the term “cancer” or “malignancy” or “tumour” means and includes all forms of cancer, including but not limited to bladder, brain, breast, cervical, colorectal, uterine, esophagus, Hodgkin lymphoma, kidney, larynx, Leukaemia, Lip, Lung, Multiple myeloma, Non-Hodgkin lymphoma, Oral cavity, Ovary, Pancreas, Prostate, Skin, Stomach, Testis, and Thyroid cancers.

In this disclosure the term “carrier” or “excipient” means and includes all suitable compositions which will be acceptable in the sense of being compatible with the other ingredients of the composition and not significantly deleterious to the recipient. The carrier or excipient can be a solid or a liquid, or both, and is preferably formulated with the compound of the invention as a unit-dose composition, for example, a tablet, which can contain from 0.05% to 95% by weight of the active compound. Such carriers or excipients include inert fillers or diluents, binders, lubricants, disintegrating agents, solution retardants, resorption accelerators, absorption agents, and coloring agents. Suitable binders include starch, gelatin, natural sugars such as glucose or .beta.-lactose, corn sweeteners, natural and synthetic gums such as acacia, tragacanth or sodium alginate, carboxymethylcellulose, polyethylene glycol, waxes, and the like. Lubricants include sodium oleate, sodium stearate, magnesium stearate, sodium benzoate, sodium acetate, sodium chloride, and the like. Disintegrators include starch, methyl cellulose, agar, bentonite, xanthan gum, and the like. Possible components of an excipient of particular embodiments may be or may comprise mannitol, pregelatinized starch, magnesium stearate, sodium saccharine, talcum, cellulose ether derivatives, gelatin, sucrose, citrate, propyl gallate, lactose, white soft sugar, sodium chloride, glucose, urea, starch, calcium carbonate, kaolin, crystalline cellulose, silicic acid, calcium silicate, potassium phosphate, cacao butter, hardened vegetable oil, kaolin, and others, all of which will be readily apparent to those skilled in the art.

In alternative embodiments the conditions treatable using the compositions and methods disclosed herein may be or may include: bacterial infections and mycoses; parasitic diseases, viral diseases (ie. HIV), musculoskeletal diseases; digestive system diseases (ie. colitis, crohn's disease, hepatitis); stomatognathic diseases (ie. mumps, periodontal disease); respiratory tract diseases (ie. asthma, bronchitis); otorhinolaryngologic (ear, nose, throat) diseases; nervous system diseases (ie. alzheimer's disease, multiple sclerosis); eye diseases (ie. glaucoma); urogenital diseases (ie. kidney disease); cardiovascular diseases (ie. heart disease, hypertension); hemic and lymphatic diseases; neonatal diseases; skin and connective tissue diseases (ie. arthritis, dermatitis, psoriasis, acne, lupus, rosacea); nutritional and metabolic diseases; endocrine diseases (ie. diabetes); immunologic diseases (ie. allergies, thyroid disorders, arthritis, anaphylaxis).

### First Embodiment

In a first embodiment there is disclosed a superparamagnetic nanoparticle or a plurality thereof which may comprise a metal oxide, may be polyhedral and may be coated with silica and may have amine groups. In embodiments the nanoparticle or the core of the nanoparticle has a shape which may be: primitive cubic, body-centered cubic, face-centered cubic, primitive tetragonal, body-centered tetragonal, primitive orthorhombic, body-centered orthorhombic, single face-centered orthorhombic, multiple face-centered orthorhombic, primitive monoclinic, single face-centered monoclinic, primitive triclinic, single face-centered hexagonal, and rhombohedral, monohedral, parallelohedral, dihedral, dishpenoid, prism, pyramid, dipyramid, trapezohedron, scalenohedron, rhombohedron, and tetrahedron. In particular embodiments the nanoparticles may be substantially cubic.

In some embodiments the metal, or one of the metals comprising, the metal oxide may be selected from the group consisting of: Cobalt, Titanium, Manganese, Magnesium, Nickel, Copper, Zinc, Vanadium, Gold and Iron. In embodiments the nanoparticle may be coated and the coating may comprise a material selected from the group consisting of silica, dextran, polyethylene glycol, polylactic acid, polyglycolic acid, and alginate. In some embodiments the nanoparticle may be functionalized.

In embodiments the nanoparticle may be made by a process comprising heating the superparamagnetic metal oxide to a temperature of greater than 50° C., 80° C., 100° C., 120° C., 140° C., 160° C., 180° C., 200° C., or greater than 200° C. In further alternative embodiments the nanoparticles may comprise superparamagnetic metal oxide and the process may further comprise autoclaving the superparamagnetic metal oxide.

In embodiments metal oxide may be comprised in a core and the nanoparticle may be coated and may be coated with silica and may be functionalized with functional groups which may comprise amine groups. In further embodiments the nanoparticle may be functionalized with a plurality of reactive groups. In further embodiments the metal oxide is comprised in a core and the nanoparticle further comprises a silica coating associated with the core. In embodiments nanoparticle may have a plurality of reactive primary amino groups. It will be understood that a nanoparticle may be functionalized with or without the incorporation of a coat and a coat may or may not be functionalized. In embodiments the nanoparticle according the nanoparticle may be conjugated to a molecule selected from the group consisting of: nucleic acid, protein, antibody, lectin, antibiotic, pharmaceutical, anti-cancer drug, diagnostic and therapeutic compounds, and may include wound healing therapeutics.

In embodiments the nanoparticles may be doped with a range of metals or may be formed using a range of metals. Those skilled in the art will readily identify all suitable metals and will readily adapt the methods disclosed to the preparation of nanoparticles comprising such metals. In embodiments nanoparticles comprised primarily of a first metal may be doped to comprise a proportion of one or more other metals. Where the primary metal is Fe, formation of the metal doped superparamagnetic nanoparticle may be accomplished buy combining trivalent Fe(III) salt with divalent salts of the desired doping metal which may, in embodiments be Cobalt, Nickel, Copper, Zinc and others. It will be understood that nanoparticles with different chemical compositions may have different polyhedral structures.

In embodiments, the compositions used may comprise iron oxide, may comprise silica, and may comprise iron oxide coated with silica. In alternative embodiments alternative metals and compounds may be used. For example but without limiting the foregoing, in certain embodiments it may be possible to replace one or more Oxygen atoms of the compositions with Nitrogen, Phosphorous, or Sulfur and it may be possible to replace one or more of the Iron atoms with alternative transition metals.

### Second Embodiment

In a Second Embodiment there is disclosed a method for synthesizing a polyhderal superparamagnetic metal oxide nanoparticle which may comprise making amorphous nanoparticles of the superparamagnetic metal oxide and heating the amorphous nanoparticles to more than about 100° C. for more than about 8 hours.

In embodiments the nanoparticle may have a shape selected from the group consisting of: primitive cubic, body-centered cubic, face-centered cubic, primitive tetragonal, body-centered tetragonal, primitive orthorhombic, body-centered orthorhombic, single face-centered orthorhombic, multiple face-centered orthorhombic, primitive monoclinic, single face-centered monoclinic, primitive triclinic, single face-centered hexagonal, and rhombohedral, monohedral, parallelohedral, dihedral, dishpenoid, prism, pyramid, dipyramid, trapezohedron, scalenohedron, rhombohedron, and tetrahedron. In embodiments the metal may be selected from the group consisting of: Cobalt, Titanium, Manganese, Magnesium, Nickel, Copper, Zinc, Vanadium, Gold, Platinum, Palladium and Iron.

In embodiments the method further comprises co-precipitating a mixture of a first metal ion and a second metal ion to make the amorphous nanoparticles. In embodiments the first metal ion and the second metal ion may be different valency states of the same metal and in particular embodiments the first metal ion may be an Fe(II) ion and the second metal ion may be an Fe(III) ion.

In embodiments the method may comprise heating the precipitate to at least about 100° C. for at least about 10 hours. In embodiments the nanoparticle may be coated with silica and/or amine groups.

In embodiments amorphous superparamagnetic nanoparticles may be prepared by conventional methods and then treated to yield polyhedral, coated, and functionalized forms any other forms disclosed as part of other embodiments. In embodiments the superparamagnetic material may be Iron and the method of preparation may be co precipitation of Fe(II) and Fe(III) ions. Many other conventional methods will be readily understood by those skilled in the art who will readily implement them to produce a primary preparation of magnetic nanoparticles which may be spherical or amorphous. In alternative embodiments the amorphous nanoparticles and/or the polyhedral nanoparticles may have a diameter of about of between about 1 and about 30 nm, or a diameter of between about 5 and about 15 nm, and any preparation of nanoparticles may comprise nanoparticles with diameters of about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nm or may have diameters of greater than about 30 nm.

In embodiments amorphous nanoparticles may be heated to generate polyhedral forms. The heating may be carried out in an autoclave in deoxygenated water and crystalline nanoparticles may be produced. Where the magnetic material is Iron, this treatment may yield polyhderal nanoparticle which may appear crystalline and may appear broadly cubic and may have an overall diameter of about 8 nm.

While in embodiments the heating may be carried out in deoxygenated water in an autoclave, alternative embodiments may comprise heating in different solvents. Oxygen may be excluded to prevent further oxidation of the nanoparticles. It will be appreciated that in embodiments the heating conditions may be varied as regards both time and temperature. For instance in alternative embodiments the temperature used may be above about 40, 50, 60, 80, 90, 100, 110, 120, 130, 140, 150, 160, 170, 180, 190, 200, 250, 300, 400, 500, 600 or more degrees celsius and may be below about 100, 110, 120, 130, 140, 150, 160, 170, 180, 190, 200, 250, 300, 400, 500, 600 degrees celsius and may fall in any range defined by such alternatives. In alternative embodiments the nanoparticles may be heated for between about 5 hours and about 10 hours, between about 10 and about 15 hours, between about 15 and about 20 hours, between about 20 and about 25 hours, up to about 30, 40, 50 or more hours. In embodiments the crude nanoparticles may be heated up to 100-120° C. for 12-24 h.

It will be understood that in embodiments the heating may be carried out simultaneously with the precipitation process and that in others it may be carried out following collection of the precipitate.

It will be understood that the polyhedral nanoparticles produced according to the methods of this embodiment may be further modified by doping, coating, functionalization, conjugation and the like using conventional methods all of which will be readily understood and implemented by those skilled in the art.

### Third Embodiment

In a further embodiment there are disclosed methods for labelling cells using nanoparticles of any of the other embodiments. The method may comprise labelling a cell with a polyhedral superparamagnetic nanoparticle; and detecting the nanoparticle. In embodiments the nanoparticle may be conjugated to chemicals or biologics, including those with selective affinity for receptors or other structures on the cell membrane, such as therapeutic antibodies, radioisotope labeled biologics, and a range of pharmaceuticals. In embodiments the cell to be labelled may be phagocytic, non phagocytic, and may be mammalian and may be non-human, human, and may be a Stem cell, a Nerve cell, a Tumoral cell, a Osteoblast, a Osteocyte, a Osteoclast, a Chondroblast, a Chondrocyte, a Myocyte, a Adipocyte, a Fibroblast, a Tendon cell, a Podocyte, a Juxtaglomerular cell, a Intraglomerular mesangial cell/Extraglomerular mesangial cell, a Kidney proximal tubule brush border cell, a Macula densa cell, a Gastric chief cell, a Parietal cell, a Goblet cell, a Paneth cell, a Enteroendocrine cells, a Enterochromaffin cell, a APUD cell, a Hepatocyte, a Kupffer cell, a Myocardiocyte, a Pericyte, a Pneumocyte (Type I pneumocyte, Type II pneumocyte), a Clara cell, a Goblet cell, a glial cells (Astrocyte, Microglia), a Thyroid epithelial cell, a Parafollicular cell, a Parathyroid chief cell, a Chromaffin cell, a lymphoid: B/T T cell, a Natural killer cell, a granulocytes (Basophil granulocyte, Eosinophil granulocyte, Neutrophil granulocyte/Hypersegmented neutrophil), a Monocyte/Macrophage, a Red blood ell(Reticulocyte), a Mast cell, a Thrombocyte/Megakaryocyte, a Dendritic cell. In embodiments the method may be applied to cells in vitro, in vivo, or ex vivo. In particular embodiments the cell may be in a mammalian body and may be in a human body. In particular embodiments the method may comprise a step selected from among: labelling the cell in vivo, labelling the cell ex vivo, injecting the nano nanoparticle, orally delivering the nanoparticles, parenterally delivering the nanoparticles, or delivering the nanoparticles by catheter. In embodiments the nanoparticle labelled cells may be delivered to an organism or tissue or cells using a medical device, and may be injected parenterally, subcutaneously, intravascularly, by catheter or by any other conventional methods. the observing the cells in vivo, observing the cells ex vivo, localizing the nanoparticles, localizing the nanoparticles using a magnetic field, and separating cells containing nanoparticles from cells not containing nanoparticles. In particular embodiments the method may comprise a step selected from among: labelling the cell in vivo, labelling the cell ex vivo, injecting the nano nanoparticle, orally delivering the nanoparticles, parenterally delivering the nanoparticles, observing the cells in vivo, observing the cells ex vivo, localizing the nanoparticles, localizing the nanoparticles using a magnetic field, and separating cells containing nanoparticles from cells not containing nanoparticles.

In particular embodiments the cells to be labelled may be mesenchymal stem cells (MSCs), and it will be appreciated that where the nanoparticles are long lived in the cells labelled, then they may be of particular value to label groups of cells that under repeated division so that labelling of the progenitor cells may be carried down to their offspring. The density of labelling of the progenitor cells will be readily adjusted by those skilled in the art to suit particular purposes.

In particular embodiments the nanoparticles may become localized in the lysosomes of the target cells but it will be readily apparent that alternative cellular localizations are possible. In some embodiments nanoparticles of particular embodiments may be produced using the methods set forth in Tan Weihong et al., U.S. Ser. No. 11/188,459 Jul. 25, 2005 (filed) on “Method of making nanoparticles” or modifications thereof which comprising applying to the nanoparticles a coating of silica using a silicating agent such as tetraethylorthosilicate (TEOS).

In embodiments cells may be labelled with nanoparticles ex vivo and then implanted into a body of a subject. In further embodiments labelling of cells with nanoparticles may be used as a histological labelling technique or may be used as an in vivo labelling technique. In embodiments the nanoparticles may be used to magnetically sort cells or biological materials by separating those with associated nanoparticles from those without associated nanoparticles.

### Fourth Embodiment

In a fourth embodiment there is disclosed a pharmaceutical composition comprising a nanoparticle or nanoparticles of other embodiments which may be conjugated or combined or associated with Doxorubicin, Taxol, Traditional Chinese Medicines, pharmaceuticals, or other reagents readily selected among and used by those skilled in the art. The nanoparticles may be coated, or conjugated or coated and conjugated as disclosed for any of the embodiments.

### Fifth Embodiment

In a fifth embodiment there is provided a method of treating a subject in need of treatment, the treatment comprising administering to the subject a polyhedral superparamagnetic nanoparticle. The nanoparticles may be made and may be modified as set out herein, and may be conjugated to a range of bioactive reagents. There is similarly disclosed the use of a superparamagnetic nanoparticle according to any one of the embodiments to manufacture a medicament for the treatment of a patient in need of said treatment. In a further embodiment there is disclosed the use of the nanoparticles of embodiments to label cells in a subject to diagnose or treat a patient requiring such diagnosis or treatment.

### EXAMPLES

The following are examples that illustrate materials, methods, and procedures for practicing the subject matter disclosed. It should be understood that the examples and embodiments described herein are for illustrative purposes only and that various modifications or changes in light thereof will be suggested to persons skilled in the art and are to be included within the spirit and purview of this application.

Materials and Methods

Pure water was bubbled with high purity nitrogen for at least 30 min before use. All chemical reactions were performed under high purity nitrogen.

For Transmission Electron Microscope (TEM) analysis, one drop of sample in ethanol was added to the carbon-coated copper grid and was allowed to evaporate to dryness. Built-in energy-dispersive X-ray (EDX) spectroscopy was performed by locating a region (˜20 nm×20 nm) with substantial amount of materials. For inductively coupled plasma-optical emission spectroscopy (ICP-OES) analysis, samples were dissolved in 2% HCl solution with a few drops of SnCl₂ solution. Iron absorption was observed at 238.204 nm.

### Example 1

Preparation of SPIO Nanoparticles

Amorphous SPIO nanoparticles were prepared by a chemical co-precipitation method with 2 equivalents of ferric chloride and 1 equivalent of ferrous chloride with aqueous sodium hydroxide solution. The co-precipitation mixture comprised 0.17 mM Fe(II), and 0.33 mM Fe(III) and to the mixture was slowly adjusted to 30 nm NaOH with shaking to yield the precipitate. The precipitate was separated by centrifugation and washes with deoxygenated water. The SPIO nanoparticles were modified by heating over 120° C. in water in an autoclave for 12 hrs to afford the cube-like SPIO nanoparticles with 6 nm in size. The precipitate was separated by centrifugation and washed with deoxygenated water twice and anhydrous ethanol for four times, then vacuum dried at 50° C. overnight to afford the re-crystallized Fe₃O₄ SPIO nanoparticles.

### Example 2

Preparation of SPIO-SiO₂ Nanoparticles

Silica-coated SPIO (SPIO-SiO₂) nanoparticles are produced by hydrolysis of tetraethylorthosilicate (TEOS) on the surfaces of the recrystallized SPIO nanoparticles. First, the cube-like SPIO nanoparticles are ultrasonically redispersed in a solution containing ethanol and water mixture. The pH value is adjusted to 9 with ammonia solution. TEOS is added dropwise under vigorous stirring, and then heated to reflux. The precipitate is separated by centrifugation and washes several times with water and ethanol to afford the SPIO-SiO₂ nanoparticles with 10 nm in size. The coating thickness can be tuned by using different amounts of TEOS.

### Example 3

Preparation of SPIO-SiO₂—NH₂ Nanoparticles

SPIO-SiO₂—NH₂ nanoparticles are produced by hydrolysis of aminopropyltriethoxysilane (APTES) on the surfaces of the cube-like SPIO nanoparticles. First, the cube-like SPIO nanoparticles are ultrasonically redispersed in a solution containing ethanol and water mixture. The pH value is adjusted to 9 with ammonia solution. APTES is added dropwise under vigorous stirring, and then heated to reflux. The precipitate is separated by centrifugation and washes several times with water and ethanol to afford the SPIO-SiO₂—NH₂ nanoparticles with 8 nm in size.

### Example 4

Preparation of SPIO-SiO₂—NH₂ Nanoparticles

SPIO-SiO₂—NH₂ nanoparticles are produced by hydrolysis of aminopropyltriethoxysilane (APTES) on the surfaces of the SPIO-SiO₂ nanoparticles. First, the 10 nm SPIO-SiO₂ nanoparticles are ultrasonically redispersed in a solution containing ethanol and water mixture. The pH value is adjusted to 9 with ammonia solution. APTES is added dropwise under vigorous stirring, and then heated to reflux. The precipitate is separated by centrifugation and washes several times with water and ethanol to afford the SPIO-SiO₂—NH₂ nanoparticles with 12 nm in size.

Characteristics of the Nanoparticles

Evaluation of the physical property of SPIO nanoparticles was carried out using transmission electron microscope (TEM), energy-dispersive X-ray (EDX) spectroscopy, inductively coupled plasma-optical emission spectroscopy (ICP-OES), X-ray diffraction (XRD) and vibrating sample magnetometer (VSM).

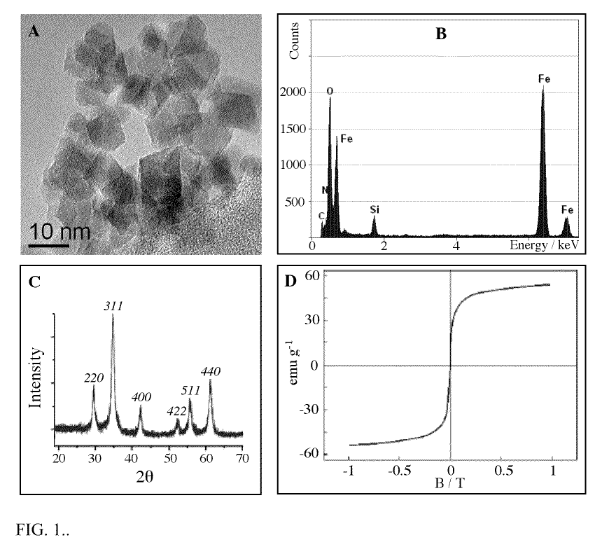

SPIO-SiO₂ and SPIO-SiO₂—NH₂ nanoparticles were characterized employing TEM, EDX, XRD and VSM. For instance, TEM analysis of the SPIO nanoparticles demonstrated that they are nearly mono-dispersed cube-like crystals, in which the SPIO-SiO₂ nanoparticles have an average size of 10.7±2.6 nm and the SPIO-SiO₂—NH₂ of 8.5±3.0 nm. By way of an example, TEM image of the SPIO-SiO₂—NH₂ (shown in FIG. 1A) reveals a single iron oxide core (dark dots) with single silica shell (thin white layer around the dark dots) structure rather than multi-core with single silica shell structure, demonstrating the success of the uniform thin silica coating deposition on each individual SPIO nanoparticles.

EDX analysis reveals that the iron contents of SPIO-SiO₂ and SPIO-SiO₂—NH₂ (shown in FIG. 1B) nanoparticles are 22.0±0.2% and 48.3±0.3% respectively, which are comparable to the values determined by ICP-OES (21.6±0.1% for SPIO-SiO₂ and 44.8±0.2% for SPIO-SiO₂—NH₂) in fixed nanoparticle concentrations. Accounting to the same iron oxide core size for both SPIO-SiO₂ and SPIO-SiO₂—NH₂ nanoparticles, a higher iron content (44.8%) for SPIO-SiO₂-NH₂ nanoparticles reveals that these nanoparticles had a thinner silica shell layer than that of the SPIO-SiO₂ nanoparticles. Moreover, EDX analysis permits the determination of silicon (Si, 10.6%), carbon (C, 11.3%) and nitrogen (N, 4.5%) elements present in the SPIO-SiO₂—NH₂ nanoparticles (FIG. 1B).

Both the XRD patterns of SPIO-SiO₂ and SPIO-SiO₂—NH₂ (shown in FIG. 1C) reveal that the nanoparticles exhibit several peaks corresponding to the characteristic interplanar spacings 220, 311, 400, 422, 511 and 400 of the spinel structure with 20, 29.5, 34.7, 42.3, 52.4, 56.1 and 61.7, respectively. These signals reveal the characteristics of magnetite core. The surface modification processes using either TEOS or APTES on the same magnetite core do not affect the original morphology and the crystallinity of the magnetite core.

FIG. 1D shows the magnetization (emg g⁻¹) versus applied magnetic field (B/T). The hysteresis loop of SPIO-SiO₂—NH₂ nanoparticles demonstrates (shown in FIG. 1D) that there is no coercive force, thus featuring a superparamagnetic behavior. The saturation magnetization of the SPIO-SiO₂—NH₂ nanoparticles is 52.5 emu g⁻¹ Fe, which is slightly less than that of the commercially available contrast agent—Feridex (˜70 emu g⁻¹ Fe). The saturation magnetization of the SPIO-SiO₂ nanoparticles is determined to be 43.5 emu g⁻¹ Fe. A high saturation magnetization is essential for the T2-weighted MRI because the spin-spin relaxation process of protons in the surrounded water molecules is facilitated by a large magnitude of magnetic spins in the nanoparticles.

FIG. 1. Shows a: TEM image of cube-like SPIO-SiO₂—NH₂ nanoparticles with an average size of 8.5 nm. The silica shell can be observed as thin, white layer around each iron oxide nanoparticle (dark dots), resulting in a single iron oxide nanoparticle core/single silica shell structure. B: EDX spectrum showing the elements (Fe, Si, O, C and N) present in the SPIO-SiO₂—NH₂ nanoparticles. C: XRD spectrum of the SPIO-SiO₂—NH₂ nanoparticles, indicating the characteristic signals attributed to the crystal lattice of magnetite core. D: VSM spectrum of the SPIO-SiO₂—NH₂ nanoparticles, revealing a superparamagnetic behavior with no coercive force in the hysteresis loop.

In our example, MR relaxometry of the SPIO nanoparticles is performed using a clinical 1.5 T whole-body MR system. With MR SPIO nanoparticles in water and in room temperature, relaxivity (r2) are determined to be 18.9±3.6 mM¹sec⁻¹ for the SPIO-SiO₂ and 43.5±9.1 mM⁻¹sec⁻¹ for the SPIO-SiO₂—NH₂ nanoparticles.

SPIO Mesenchymal Stem Cell Labelling

20-week-old male New-Zealand white rabbits with body weight of 3.5-4 kg are used. Bone marrow is aspirated from rabbit iliac bone with an 18G BD syringe. Bone marrow is washed with Dubellco modified eagle medium (DMEM, Gibco 31600) (bone marrow:DMEM=1:4). The mixture is spinned. Then the fat debris and supernatant is removed. The pellet is resuspended with 10% FCS (Fetal Calf Serum, Gibco 16140) in DMEM. The cell suspension is transferred to 75 cm² tissue culture flask. The cell culture is incubated at 37° C. with 5% CO₂. Half of the basal medium was refreshed after 4 days and all the culture medium is refreshed after another 3 days. The adherent mesenchymal stem cells (MSCs) are grown in colony. The cells can be subcultured into other culture flask for cell expansion after 5-7 days.

As an example, rabbit bone marrow derived MSCs are incubated with SPIO-SiO₂ or SPIO-SiO₂—NH₂ nanoparticles at serum free DMEM culture medium with fixed iron concentrations for 18 hrs, with iron concentrations of 4.5 μg/mL. Immediately before labelling, SPIO nanoparticles are sonicated for 15 min. Following the above procedure, SPIO nanoparticles are labelled into MSCs. To confirm the labelling efficiency, Prussian blue staining can be performed, where after fixation with 2.5% glutaraldehyde (Sigma G4004), the cells are incubated with 1% potassium ferrocyanide (Sigma P3289) and 2% HCl for 10-15 min, then 1% neutral red (Sigma N8002) is added to stain nuclear.

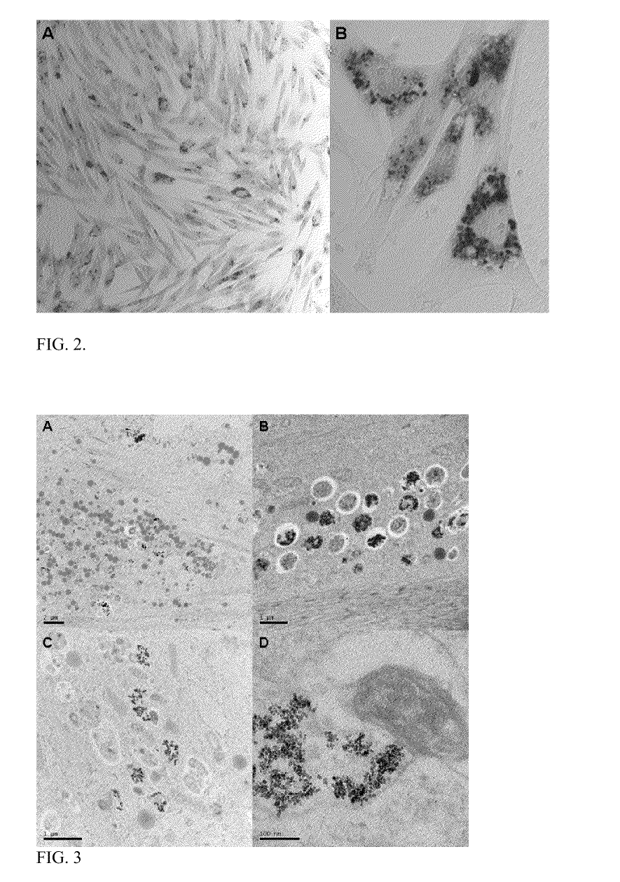

Prussian blue staining showed MSCs labelling efficiency can be achieved in 100% using both the SPIO nanoparticles (SPIO-SiO₂ and SPIO-SiO₂—NH₂). All MSCs incorporated numerous SPIO nanoparticles (FIG. 2). After labelling with both SPIO-SiO₂ and SPIO-SiO₂—NH₂ nanoparticles, TEM demonstrated that these nanoparticles are located in the lysosomes and vesicles, but not found in the nucleus or other structures. With apparent normal nuclear morphology, apoptosis and necrosis changes are not observed (FIG. 3).

FIG. 2. shows optical microscopy images of the MSCs with Prussian blue staining, demonstrating the SPIO-SiO₂—NH₂ nanoparticle distribution within MSCs (original magnification: 200×). Figure A shows a 100% labelling efficiency. MSCs appear as normal cell morphology. Figure B shows numerous SPIO nanoparticles in four MSCs.

FIG. 3. shows TEM images (A-D) showing that the numerous SPIO-SiO₂—NH₂ nanoparticles distribute in lysosomes and vesicles of MSCs while not found in the nucleus and other supermicrostructures (A: 4200× B: 11500× C: 16500×, D: 160000×). Mono-dispersed SPIO-SiO₂—NH₂ nanoparticles remain well separated within MSCs. Cells have apparently normal nuclear morphology, and apoptosis and necrosis are not observed.

MSCs are assessed 33 days after SPIO nanoparticle labelling, which is seven MSCs normal passages after labelling, are cultured at 37° C. with 5% CO₂ without additional intervention and normal divisions are permitted. 33 days post labelling, TEM shows that both SPIO-SiO₂ and SPIO-SiO₂—NH₂ nanoparticles exist in the lysosomes and vesicles of the labelled MSCs though with less quantity compared to day 1.

ICP-OES is used to quantify the iron content. Rabbit MSCs are labelled with SPIO-SiO₂ or SPIO-SiO₂—NH₂ nanoparticles as described above. After washing with cultured medium, 100,000 cells are placed into Eppendorf tubes respectively. The cell pellets are dissolved in 2% HCl aqueous solution with a few drops of concentrated SnCl₂ solution for which the total iron concentration is within 10 to 40 ppm. Iron absorption is observed at 238.204 nm. A calibration curve is plotted by using a set of FeCl₃ standard diluted solutions. ICP-OES shows that immediately post labelling, the total iron content of MSCs labelled with SPIO-SiO2 is 17.4±3.9 pg/cell while those labelled with SPIO-SiO2-NH2 are 68.7±11.2 pg/cell.

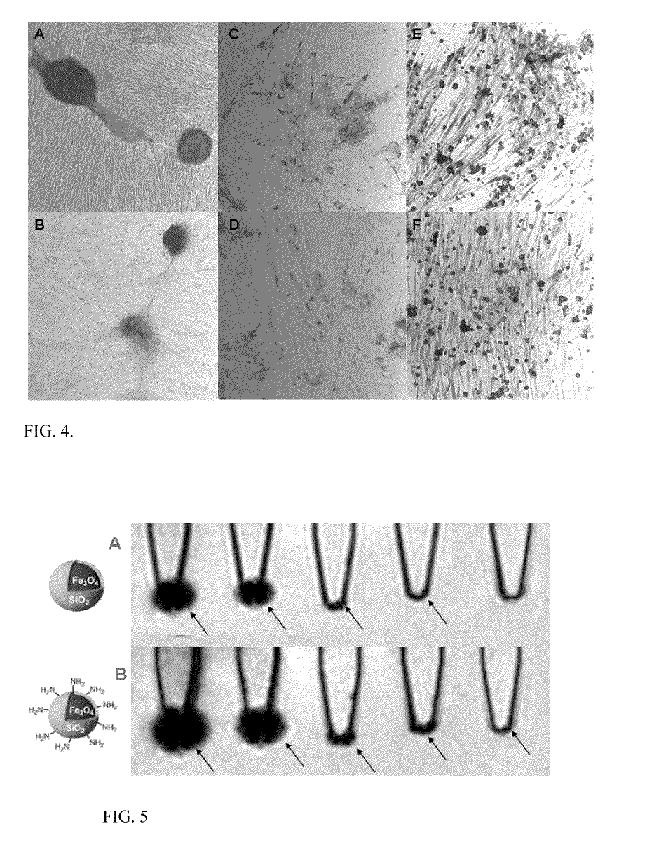

After SPIO nanoparticles labelling as described above, i.e. 4.5 μgFe/mL for 18 hrs, Trypan blue exclusion assay (Sigma T6146) is performed to assess the viability of MSCs. To assess cell growth post labelling, MSCs are cultured in 96-well plate at the density of 5000 cell/well incubated with DMEM including 10% FCS. After standard labelling procedure, SPIO nanoparticles are removed from the plate and PBS is used to rinse the residual iron nanoparticles. Fresh DMEM including 10% FCS is added again for normal growth. 3-[4,5-dimethylthiazol-2-yl]-2,5-diphenyl tetrazolium bromide (MTT) assay (Roche Molecular Biochemicals, Indianapolis, Ind.) is performed to detect MSCs growth labelled with SPIO nanoparticles. The differentiation potentials for MSCs post SPIO nanoparticles labelling are assessed as follow: 1) for osteogenic differentiation, MSCs labelled with SPIO nanoparticles are incubated with ascorbic acid, β-glycerophosphate, and dexamethasone. Four weeks after induction, alizarin red staining is performed to detect calcium nodules; 2) for adipogenic differentiation, MSCs labelled with SPIO nanoparticles are incubated dexamethasone, insulin, isobutyl methyl xanthine, and indomethacin for 4 weeks. Then oil red O staining is performed to detect the lipid vacuoles in the MSCs; 3) for chondrogenic differentiation, MSCs labelled with SPIO nanoparticles are cultured in the presence of transforming growth factor-β for 4 weeks, and then Toluidine blue staining is performed to detect glycosaminoglycans expression. Trypan blue exclusion assay results demonstrate that the cell viability is 95.7±2% and 94.2±3% after the labelling with SPIO-SiO₂ and SPIO-SiO₂—NH₂ respectively. No MSCs apparent growth inhibition is observed after the SPIO nanoparticle labelling. After both SPIO-SiO₂ and SPIO-SiO₂—NH₂ nanoparticle treatment, the osteogenic, adipogenic and chondrogenic differentiation potentials of MSCs are retained (FIG. 4).

FIG. 4. Shows the effects of SPIO-SiO₂—NH₂ on osteogenic, chondrogenic and adipogenic differentiation of rabbit bone marrow derived MSCs. MSCs are first treated with 4.5 μg[Fe]/mL SPIO-SiO₂—NH₂ for 18 hrs and then incubate with osteogenic (B, 100×), adipogenic (D, 200×) and chondrogenic (F, 200×) medium respectively for 4 weeks. A, C, and D are control MSCs without SPIO treatment for B, D and E respectively. Similar to control MSCs after induction agents, MSCs labelled with SPIO-SiO₂—NH₂ demonstrate osteogenic (B), adipogenic (D) and chondrogenic (F) differentiation.

Toxicity due to SPIO may be linked to the labelling concentration, and this concentration should be optimized for individual particular applications.

In vitro MR imaging is performed with SPIO nanoparticle-labelled MSC pellets twice, immediately and 33 days after the labelling procedure. Rabbit MSCs are labelled with SPIO-SiO₂ or SPIO-SiO₂—NH₂ nanoparticles as described above. After washing with the cultured medium, 10⁵, 5×10⁴, 10⁴, 5×10³, and 10³ cells are placed separately into 1.5 mL Eppendorf tubes. After centrifugation, Eppendorf tubes are placed perpendicular to the main magnetic induction field (BO) in a 20×12×8 cm water bath. MRI is performed on with a 3.0-T clinical whole-body MR unit (Achieva; Philips Medical Systems, Best, the Netherlands) using a transmit-receive head coil. MR sequence is a 2D gradient echo sequence with TR/TE=400/48 msec, flip angle=18, matrix=512×256, resolution=0.45×0.45 mm, slice thickness=2 mm and NEX=2. Sagittal images are obtained sectioned through the bottom tips of the Eppendorf tubes. Following MSCs labelling with both SPIO nanoparticles, substantial negative contrast (dark MR signal) is observed with cell pellets of 10⁵ and 5×10⁴ cells leading to ‘ballooning’ effect. The dark areas of this ‘ballooning’ effect from cell pellets labelled with SPIO-SiO₂—NH₂ nanoparticles have 1.8-1.9 times the size of that labelled with SPIO-SiO₂ nanoparticles at the concentration of 10⁵ and 5×10⁴ respectively. For MSCs labelled with SPIO-SiO₂ nanoparticles, pellets of ≧5000 MSCs are detectable, while MSCs labelled with SPIO-SiO₂—NH₂ nanoparticles, pellets of ≧1000 MSCs are detectable (FIG. 5). 33 days post MSCs labelling with the SPIO nanoparticles, noticeable negative contrast is observed with cell pellets ≧5×10⁴ cells labelled with SPIO-SiO₂ nanoparticles. On the other hand, noticeable negative contrast is observed with pellets ≧10⁴ MSCs labelled with SPIO-SiO₂—NH₂ nanoparticles. Please note the MRI scan techniques described hereby may not be optimal for detecting minimal amount of SPIO labelled stem cells.

FIG. 5. Shows gradient echo MR images of the MSCs pellets post SPIO nanoparticles labelling in Eppendorf tubes with culture medium. The cell number in Eppendorf tubes (from left to right) is 1×10⁵, 5×10⁴, 10⁴, 5,000, and 1,000. The MSCs in row A are labelled with SPIO-SiO₂ while the MSCs in row B are labelled with SPIO-SiO₂—NH₂ nanoparticles. For the negative contrast (dark) signal of MSC pellets labelled with SPIO-SiO₂—NH₂ nanoparticles, the area is approximately 2 times larger compared to that of the MSC pellets labelled with SPIO-SiO₂ nanoparticles. The MSC pellets are visible ≧5,000 cells with SPIO-SiO₂ labelling and ≧1000 cells with SPIO-SiO₂—NH₂ labelling.

Longitudinal Monitoring of SPIO Labelled MSCs Implanted in the Rabbit Brain

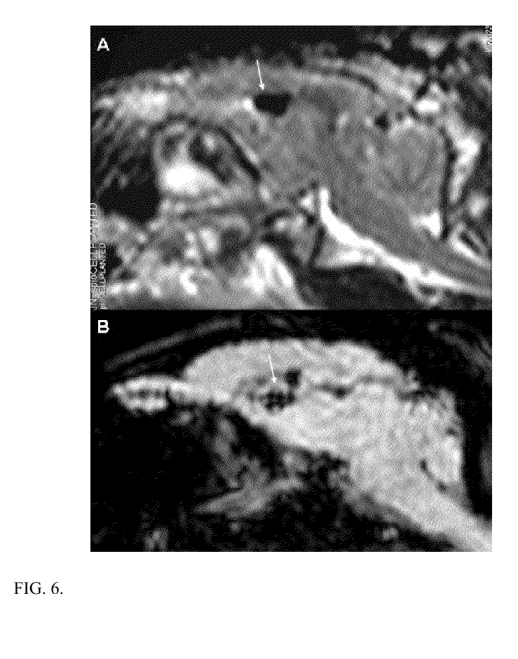

Rabbit MSCs are labelled with SPIO-SiO₂—NH₂ nanoparticles as described above. SPIO-SiO₂—NH₂ nanoparticles labelled MSCs at the number of (1×10⁵) are implanted into right hemisphere of the brain of a New Zealand male white rabbit. MRI of the rabbit brain is performed every two weeks to monitor the SPIO-SiO₂-NH₂ nanoparticles labelled MSCs. After 8 or 12 weeks, the rabbit is humanely killed and the brain is harvested for histology, which included HE staining and Prussian blue staining. MRI was performed with a 3.0-T clinical whole-body MR unit (Achieva; Philips Medical Systems, Best, the Netherlands) using a knee coil. The acquisition parameters for brain imaging included 2D gradient echo sequences, TR/TE=328/16 msec, FOV=80*80 mm, flip angle=18, in-plane actual resolution 0.29*0.37 mm with apparent resolution 0.16*0.16 mm, slice thickness=1 mm, NEX=10. SPIO-SiO₂—NH₂ nanoparticles labelled MSCs induced signal void on 2D gradient echo T2W images 2 days post-implantation in the brain. 8-12 weeks post MSCs implantation, signal voids at the same location is still apparently visible, though there is a slightly decrease in size (FIG. 6, 7). Histology sections with Prussian blue staining suggested SPIO nanoparticles predominantly located within stem cells and stem cell derived cells. No apparent phagocytic cells existed in the implanted sites in the brain.

FIG. 6. Shows a: Rabbit brain T2W 2D spin echo image in sagittal plane. FIG. 6A shows such a section immediately post MSCs implantation. The SPIO-labelled MSCs induce signal void area (arrow). FIG. 6B: shows the same rabbit brain with T2W 3D veno-BOLD imaging in sagittal plane obtained 8 weeks post implantation. The signal void areas induced by the SPIO-labelled MSCs are visualized (arrow).

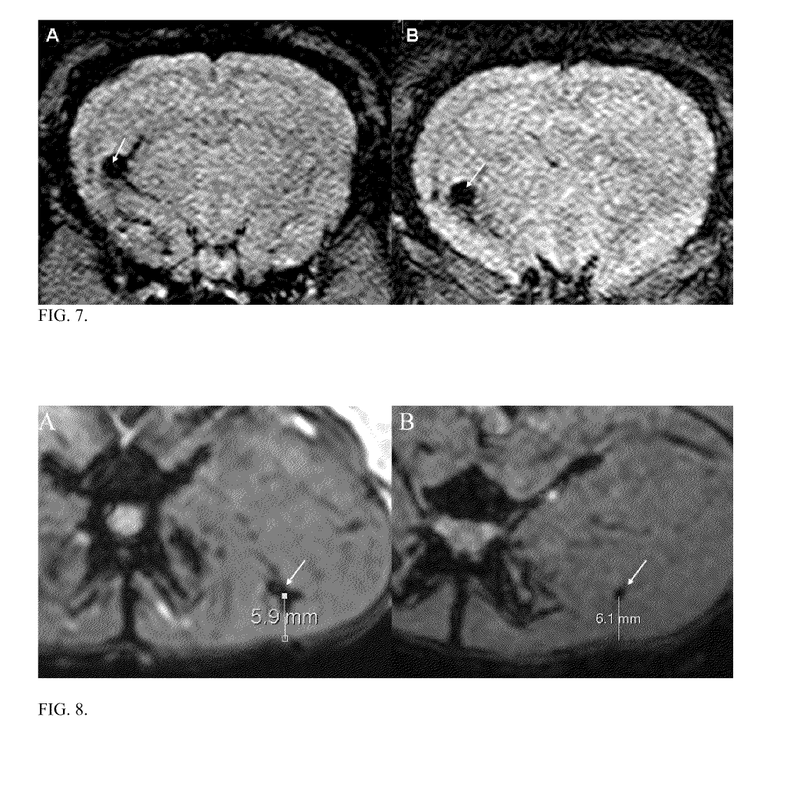

FIG. 7. Shows SPIO-SiO₂—NH₂ nanoparticles labelling of MSCs showing an induced signal void on 2D gradient echo T2W images. FIG. 7A shows labelling 2 days post-implantation in the brain. FIG. 7B shows labelling 12 weeks post MSCs implantation, signal voids at the same location is apparently visible, though there is a slight decrease in size.

Longitudinal Monitoring of SPIO Labelled MSCs Implanted in the Rabbit Erector Spinae.

Rabbit MSCs are labelled with SPIO-SiO₂—NH₂ nanoparticles as described above. SPIO-SiO₂—NH₂ nanoparticles labelled MSCs at the number of 5×10⁴ are implanted into the left erector spinae of a New Zealand male white rabbit. MRI of the left erector spinae of the rabbit is performed every two weeks to monitor the SPIO-SiO₂—NH₂ nanoparticles labelled MSCs. After 12 weeks, the rabbit is humanely killed and the left erector spinae is harvested for histology, which included HE staining and Prussian blue staining. MRI was performed with a 3.0-T clinical whole-body MR unit (Achieva; Philips Medical Systems, Best, the Netherlands) using a knee coil. The acquisition parameters for bilateral erector spinae imaging includes 2D gradient echo sequences, TR/TE=930/16 msec, FOV=80*80 mm, flip angle=18, in-plane actual resolution 0.8*0.8 mm with apparent resolution 0.5*0.5 mm, slice thickness=0.8 mm, NEX=8. SPIO-SiO₂—NH₂ nanoparticles labelled MSCs induced signal void on 2D gradient echo T2W images 2 days post-implantation in the left erector spinae. 12 weeks post MSCs implantation, signal voids at the same location was still visible, though there is a decrease in size (FIG. 8). Histology sections with Prussian blue staining suggested SPIO nanoparticles predominantly located within stem cells and stem cell derived cells. No apparent phagocytic cells exist in the implanted sites in the erector spinae muscles.

FIG. 8. Shows SPIO-SiO₂—NH₂ nanoparticles labelled MSCs induced signal void on 2D gradient echo T2W images. FIG. 8A is an image 2 days post-implantation in the left erector spinae. FIG. 8B is an image taken 12 weeks post MSCs implantation.

SPIO Labelled MSCs Implanted in the Rabbit Erector Spinae and SPIO used a Histology Marker.

Rabbit MSCs are labelled with SPIO-SiO₂—NH₂ nanoparticles as described above. SPIO-SiO₂—NH₂ nanoparticles labelled MSCs at the number of 5×10⁴ are implanted into the left erector spinae of a New Zealand male white rabbit. After 12 weeks, the rabbit is humanely killed and the left erector spinae is harvested for histology, which included HE staining and Prussian blue staining. HE plus Prussian blue staining is able to demonstrated the iron containing cells expected to be derived from implanted stem cells.

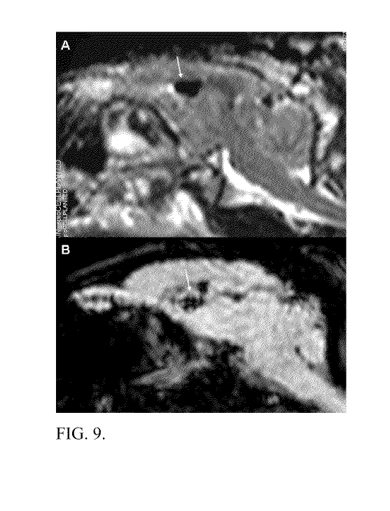

FIG. 9A is a light microscope view of MSCs 3 week after labelling with SPIO-SiO₂—NH₂. FIG. 9B is an equivalent view to FIG. 9A but shows MSCs 3 weeks after labelling with PEG-6000 coated SPIO. MSCs in 9A and 9B have similar initial iron loading at day 0.

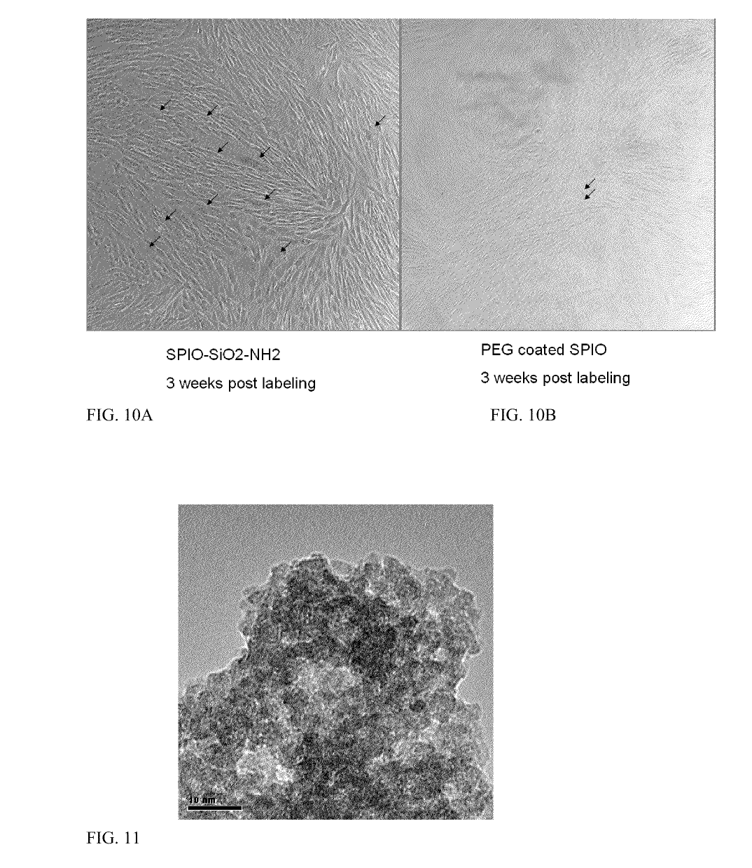

FIG. 10. is a microscope view of 12 weeks post SPIO-SiO₂—NH₂ nanoparticles labelled MSCs implantation in the left erector spinae of a rabbit. HE & Prussian blue double staining demonstrate iron (SPIO) containing cells. These cells are considered to be derived from the implanted MSCs and differentiated to smooth muscle cells. FIG. 10A is a light microscope view of MSCs 3 week after labelling with SPIO-SiO₂—NH₂. FIG. 10B is an equivalent view to FIG. 10A but shows MSCs 3 weeks after labelling with SPIO-SiO₂—NH₂.

FIG. 11 is a TEM view of nanoparticles (Fe₃O₄—NH₂, without silica coating) according to an embodiment.

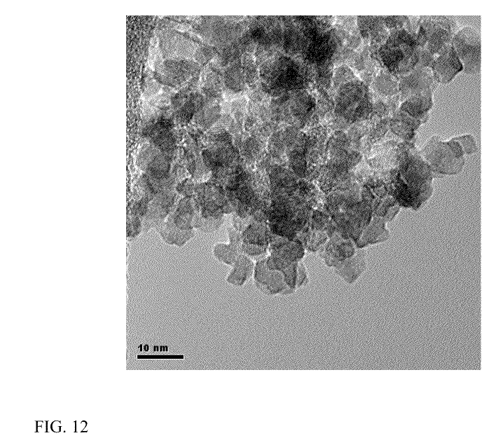

FIG. 12 is a TEM view of nanoparticles (MnFe₂O₄—NH₂, without silica coating) according to an embodiment.

The embodiments and examples presented herein are illustrative of the general nature of the subject matter claimed and are not limiting. It will be understood by those skilled in the art how these embodiments can be readily modified and/or adapted for various applications and in various ways without departing from the spirit and scope of the subject matter disclosed claimed. The claims hereof are to be understood to include without limitation all alternative embodiments and equivalents of the subject matter hereof. Phrases, words and terms employed herein are illustrative and are not limiting. Where permissible by law, all references cited herein are incorporated by reference in their entirety. It will be appreciated that any aspects of the different embodiments disclosed herein may be combined in a range of possible alternative embodiments, and alternative combinations of features, all of which varied combinations of features are to be understood to form a part of the subject matter claimed.

The embodiments of the invention in which an exclusive property or privilege is claimed are defined as follows:

1. A polyhedral superparamagnetic nanoparticle comprising a metal oxide.

2. The polyhedral superparamagnetic nanoparticle according to claim 1 wherein the nanoparticle has a shape selected from the group consisting of: primitive cubic, body-centered cubic, face-centered cubic, primitive tetragonal, body-centered tetragonal, primitive orthorhombic, body-centered orthorhombic, single face-centered orthorhombic, multiple face-centered orthorhombic, primitive monoclinic, single face-centered monoclinic, primitive triclinic, single face-centered hexagonal, and rhombohedral, monohedral, parallelohedral, dihedral, dishpenoid, prism, pyramid, dipyramid, trapezohedron, scalenohedron, rhombohedron, and tetrahedron.

3. The superparamagnetic nanoparticle according to claim 1 wherein the metal is selected from the group consisting of: Cobalt, Titanium, Manganese, Magnesium, Nickel, Copper, Zinc, Vanadium, Gold, Palladium, Platinum and Iron.

4. The superparamagnetic nanoparticle according to claim 1 wherein the nanoparticle is coated and the coating comprises a material selected from the group consisting of silica, dextran, polyethylene glycol, polylactic acid, polyglycolic acid, poly(N-isopropylacrylamide) (PNIPAM), hydroxyapatite, layered double hydroxide and alginate.

5. The superparamagnetic nanoparticle according to claim 1 wherein the nanoparticle is functionalized with functional groups selected from the group consisting of amine, ammonium, alkylamine, dialkylamine, amide, hydroxyl, ether, carboxyl, ester, thiol, thioether, alkene, and alkyne.

6. The superparamagnetic nanoparticle according to claim 1 wherein the nanoparticle is made by a process comprising heating the superparamagnetic metal oxide to above a temperature of greater than 50° C., 80° C., 100° C., 120° C., 140° C., 160° C., 180° C., 200° C., or greater than 200° C.

7. The nanoparticle according to claim 1 wherein the nanoparticle comprises superparamagnetic iron oxide.

8. The nanoparticle according to claim 3 wherein said nanoparticle is substantially cubic.

9. The nanoparticle of claim 2 wherein said nanoparticle has a diameter in a range selected from the group consisting of: between about 1 nm and about 500 nm, between about 1 nm and about 300 nm, between about 1 nm and about 150 nm, between about 1 nm and about 50 nm, between about 1 nm and about 10 nm, between about 3 and about 10 nm and between about 5 nm and about 8 nm.

10. The nanoparticle according to claim 9 wherein the nanoparticle is conjugated with a group that comprises a molecule selected from the group consisting of: nucleic acid, protein, antibody, lectin, carbohydrate, antibiotic, pharmaceutical, anti-cancer drug, and wound healing drug.

11. A method for synthesizing a polyhedral superparamagnetic metal oxide nanoparticle, said method comprising the steps of:
making amorphous nanoparticles of the superparamagnetic metal oxide and heating the amorphous nanoparticles to more than about 100° C. for more than about 8 hours.

12. The method according to claim 11 wherein the nanoparticle has a shape selected from the group consisting of: primitive cubic, body-centered cubic, face-centered cubic, primitive tetragonal, body-centered tetragonal, primitive orthorhombic, body-centered orthorhombic, single face-centered orthorhombic, multiple face-centered orthorhombic, primitive monoclinic, single face-centered monoclinic, primitive triclinic, single face-centered hexagonal, and rhombohedral, monohedral, parallelohedral, dihedral, dishpenoid, prism, pyramid, dipyramid, trapezohedron, scalenohedron, rhombohedron, and tetrahedron.

13. The method according to claim 11 further comprising coating the nanoparticles with a material comprising an ingredient selected from the group consisting of silica, dextran, polyethylene glycol, polylactic acid, polyglycolic acid, poly(N-isopropylacrylamide) (PNIPAM), hydroxyapatite, layered double hydroxide and alginate.

14. The method according to claim 11 comprising heating the precipitate to at least about 100 C for at least about 10 hours under pressure.

15. A pharmaceutical composition comprising the nanoparticle according to claim 1 in association with a pharmaceutically acceptable carrier.

16. A method of labelling a cell comprising:
(c) labelling said cell with a nanoparticle according to claim 1; and
(d) detecting said nanoparticle.

17. The method according to claim 16 wherein the nanoparticle has a shape selected from the group consisting of: primitive cubic, body-centered cubic, face-centered cubic, primitive tetragonal, body-centered tetragonal, primitive orthorhombic, body-centered orthorhombic, single face-centered orthorhombic, multiple face-centered orthorhombic, primitive monoclinic, single face-centered monoclinic, primitive triclinic, single face-centered hexagonal, and rhombohedral, monohedral, parallelohedral, dihedral, dishpenoid, prism, pyramid, dipyramid, trapezohedron, scalenohedron, rhombohedron, and tetrahedron.

18. The method according to claim 16 wherein the metal is selected from the group consisting of: Cobalt, Titanium, Manganese, Magnesium, Nickel, Copper, Zinc, Vanadium, Gold, Palladium, Platinum and Iron.

19. A method of treating a subject in need of said treatment, said treatment comprising administering to said subject a superparamagnetic nanoparticle according to claim 1 conjugated with a therapeutically effective group and in association with a pharmaceutically acceptable excipient.

20. The use of a superparamagnetic nanoparticle according to claim 1 to manufacture a medicament.
